(19) United States
(12) Patent Application Publication
(10) Pub. No.: US 2022/0218848 A1
(43) Pub. Date: Jul. 14, 2022

(54) BONE-SPECIFIC COMPLEX COMPRISING BONE CELL TRANSFORMATION RECOMBINANT PROTEIN AND BONE-TARGETING MOLECULE

(71) Applicant: SEOUL NATIONAL UNIVERSITY R&DB FOUNDATION, Seoul (KR)

(72) Inventors: Nathaniel Suk-Yeon HWANG, Seoul (KR); Gwangli PARK, Busan (KR)

(21) Appl. No.: 17/605,283

(22) PCT Filed: Apr. 23, 2020
(86) PCT No.: PCT/KR2020/005353

§ 371 Date: Oct. 21, 2021

(30) Foreign Application Priority Data
Apr. 26, 2019 (KR) 10-2019-0049370

(51) Int. Cl. A61K 49/00; C07K 14/47

(52) U.S. Cl. CPC A61K 49/0056; C07K 14/4702; C07K 2319/60; A61K 49/0032; C07K 2319/33; C07K 14/47

(57) ABSTRACT

The present invention relates to a bone-specific complex and can solve the stability problem of a viral carrier and foreign gene expression technology by inducing overexpression of targeted endogenous genes without the introduction of foreign genes. The present invention allows enhanced efficiency by stably labeling a targeted object and accurately confirming whether or not application is carried out to the targeted object, and thus can efficiently be applied to the field of developing a new protein-based drug technology requiring particular cell transformation such as osteoporosis and direct transdifferentiation.

### TECHNICAL FIELD

The present invention relates to a bone-specific complex comprising a bone cell transformation recombinant protein and a bone-targeting molecule.

In addition, the present invention relates to a method for preparing and applying the complex.

### BACKGROUND ART

In regenerative medicine, research is continuing to replace or regenerate human cells, tissues or organs to restore normal function. A research subject includes cells, drugs, materials, and medical devices, and in particular, research on autologous cells is focused in order to solve immune rejection phenomenon. In addition, as progressing toward ageing societies at an accelerated rate, research and attempts are being made on agents that can be usefully used as cell therapy for musculoskeletal disorders related to bones, cartilages, skeletal muscles, ligaments, tendons, and so on.

However, when an inducing factor is injected for differentiation of a particular tissue in vivo, cell transformation may generally occur due to unintended random delivery and there is concern about progression to cancer cells. Specifically, as a cell transformation technology, transfection of foreign genes into cells via viral vectors is traditionally applied. However, the viral vectors and foreign gene expression need to be improved in terms of safety. In addition, direct transdifferentiation with a virus is applied locally and has a limitation in that it is difficult to apply it in practice due to low efficiency of cellular conversion.

Therefore, in order to stably induce expression on target tissues without introduction of foreign genes, it is necessary to study a substance targeted for delivery to a particular tissue.

### DETAILED DESCRIPTION OF THE INVENTION

### Technical Problem

An object of the present invention is to provide a bone-specific complex comprising a bone cell transformation recombinant protein and a bone-targeting molecule.

Another object of the present invention is to provide a method for preparing and using the complex.

### Solution to Problem

In order to solve the problems, the present invention provides a bone-specific complex comprising a bone cell transformation recombinant protein and a bone-targeting molecule.

According to an embodiment, the bone cell transformation recombinant protein may comprise Oct4 protein.

According to an embodiment, the bone cell transformation recombinant protein may comprise a cell permeable protein, and the cell permeable protein may comprise 30Kc19.

According to an embodiment, the bone-targeting molecule may be a phosphonated near-infrared fluorescent substance, in particular P800SO3.

According to an embodiment, the complex may be P800SO3-30K-Oct4.

According to other aspect of the present invention, the present invention provides a method for producing a bone-specific complex, comprising:

binding a bone cell transformation inducing factor to a cell permeable protein to prepare a bone cell transformation recombinant protein;

succinimidyl ester (NHS ester) activating on a bone-targeting molecule; and

binding the activated bone-targeting molecule to the recombinant protein.

According to an embodiment, an average of 2.5 bone-targeting molecules may be synthesized onto said recombinant protein molecule.

According to an embodiment, the bone cell transformation inducing factor may comprise Oct4 protein, the cell permeable protein may comprise 30Kc19, and the bone-targeting molecule may comprise P800SO3.

The specific details of other embodiments of the present invention are included in the detailed description below.

### Effect of the Invention

By inducing overexpression of targeted endogenous genes without the introduction of foreign genes, it is possible to solve the stability problem of a viral carrier and foreign gene expression technology. In addition, it is possible to enhance efficiency by stably labeling a targeted object and accurately confirming whether or not application is carried out to the targeted object, and thus can efficiently be applied to the field of developing a new protein-based drug technology requiring particular cell transformation such as osteoporosis and direct transdifferentiation. In addition, it can be applied to determine the pharmacokinetics and pharmacodynamic effects of drugs on the bone microenvironment, and thus can be applied to real-time monitoring and diagnosis and image-guided surgery.

### BRIEF DESCRIPTION OF DRAWINGS

FIG. 1 is a schematic diagram showing a synthesis process of a bone-specific complex.

FIG. 2 is a graph showing spectrophotometer measurement results for P800SO3.

FIG. 3 is a graph showing spectrophotometer measurement results for 30Kc19-Oct4.

FIG. 4 is a graph showing spectrophotometer measurement results for P800SO3-30K-Oct4.

FIG. 5 is a color image and a fluorescence image of P800SO3-30K-Oct4.

FIG. 6 depicts administration of the complex and imaging process.

FIGS. 7 and 8 are images showing biodistribution of the complex.

FIG. 9 is a graph showing blood concentration of the complex over time.

FIG. 10 is a photograph showing the results of immunohistochemistry of the spine after complex administration.

### BEST MODE FOR CARRYING OUT THE INVENTION

Since various modifications and variations can be made in the present invention, particular embodiments are illustrated in the drawings and will be described in detail in the detailed description. It should be understood, however, that the invention is not intended to be limited to the particular embodiments, but includes all modifications, equivalents, and alternatives falling within the spirit and scope of the invention. In the following description of the present invention, detailed description of known functions will be omitted if it is determined that it may obscure the gist of the present invention.

Hereinafter, the bone-specific complex according to an embodiment of the present nvention will be described in more detail.

As used herein, the term “bone-targeting molecule” may be used interchangeably with “bone-specific molecule”, and refers to a molecule with high specificity to bone cells or osteocytes. High specificity can be confirmed by high expression of the target molecule in bone cells or osteocytes.

According to one embodiment, the bone cell transformation recombinant protein may comprise a stern cell transcription factor. For example, it may include one or more selected from the group consisting of Oct4, Sox2, Klf4, C-Myc and RUNX2.For example, the inclusion of Oct4 protein can effectively induce overexpression of endogenous genes.

According to one embodiment, the bone cell transformation recombinant protein may comprise a cell permeable protein, for example, 30Kc19, TAT and arginine-rich peptides.

According to one embodiment, the bone-targeting molecule is a material that exhibits high target signals in bones but hardly exhibits non-specific absorption in other tissues or organs and contains a phosphonated near-infrared fluorescent material. Specifically, it may include P800SO3, for example. P800SO3 has high binding affinity to bone cells, so it has high specificity to bone tissues. The intramolecular sulfonate groups enhance solubility in aqueous media, and due to their strong electron withdrawing effects, it can increase the ionization of hydroxyl groups on the fluorophores.

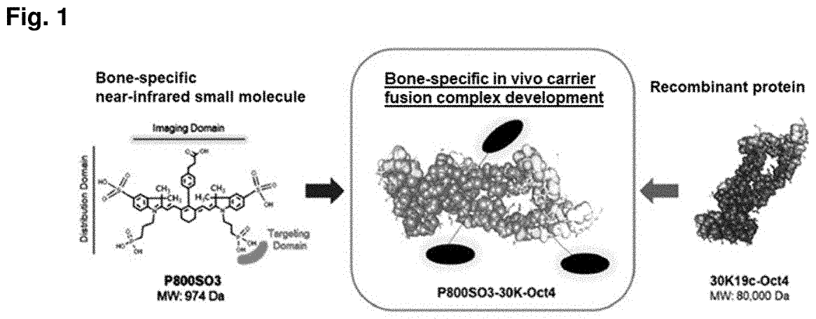

According to one embodiment, the bone-specific complex of the present invention may be P800SO3-30K-Oct4 containing Oct4 protein, 30Kc19 protein, and P800SO3, and the synthesis process is shown in FIG. 1.

According to other aspect of the present invention provides a method for producing a bone-specific complex, comprising:

binding a bone cell transformation inducing factor to a cell permeable protein to prepare a bone cell transformation recombinant protein;

succinimidyl ester (NHS ester) activating on a bone-targeting molecule; and

binding the activated bone-targeting molecule to the recombinant protein.

By activating the bone-targeting molecule with succinimidyl ester group to form an amine group (NH₂) as a binding reactive group, the activated bone-targeting molecule and the recombinant protein can be bound while minimizing damage to the function of the recombinant protein molecule.

According to one embodiment, the complex prepared according to the above method may be optimized so that an average of 1 to 5, for example, 2 to 3, for example, 2.5 bone-targeting molecules are synthesized onto the recombinant protein molecule.

Hereinafter, embodiments of the present invention will be described in detail so that those of ordinary skill in the art can easily carry out the present invention. However, the present invention may be embodied in various different forms and is not limited to the embodiments described herein.

Example 1: Preparation of bone-specific complex

Example 1-1: Activation of bone-targeting molecule

In order to prepare a complex comprising a bone cell transformation inducing factor and a bone-targeting molecule, a phosphonated near-infrared fluorescent substance, P800SO3, was used as the bone-targeting molecule. To facilitate the binding of bone cell transformation inducing factor to P800SO3 without damage, succinimidyl ester (NHS ester) was activated. The composition used for activation is shown in Table 1.

TABLE 1

| | MW | Qn (mg) | mol (nmol) | Ratio |
|---|---|---|---|---|
| P800SO3 | 974 | 1 | 1000 | 1 |
| DIEA | 129.24 | 0.5 ul | 300 | 3 |
| HSPyU | 411.28 | 2 | 500 | 10 |

For activation, specifically, 1 mg of P800SO3 was dissolved 1 mL of dimethyl sulfoxide (DMSO) in a 15 mL tube, and then 0.5 μL of diisopropylethylamine (DIEA) was added, and if necessary, a total of 2 μL of DIEA was added to bring the pH to 8.5.

4 mg of dipyrrolidino(N-succinimidyloxy)carbenium hexafluorophosphate

(HSP_{y}U) was added to the tube and stirred vigorously. After 15 minutes, it was confirmed that the pH of the intermediate reaction solution reached about 8.5, and, if necessary, DIEA was additionally added. After the reaction was completed, 10 mL of ethyl acetate was added to the solution. The precipitate was collected, and the addition of 10 mL of ethyl acetate was repeated 3 times. Finally, the collected precipitate was dried under vacuum overnight and stored in a desiccator. It was dissolved in 50 μL of DMSO immediately before use. The functional group (NHS ester) activation process of P800SO3 is shown in Scheme 1.

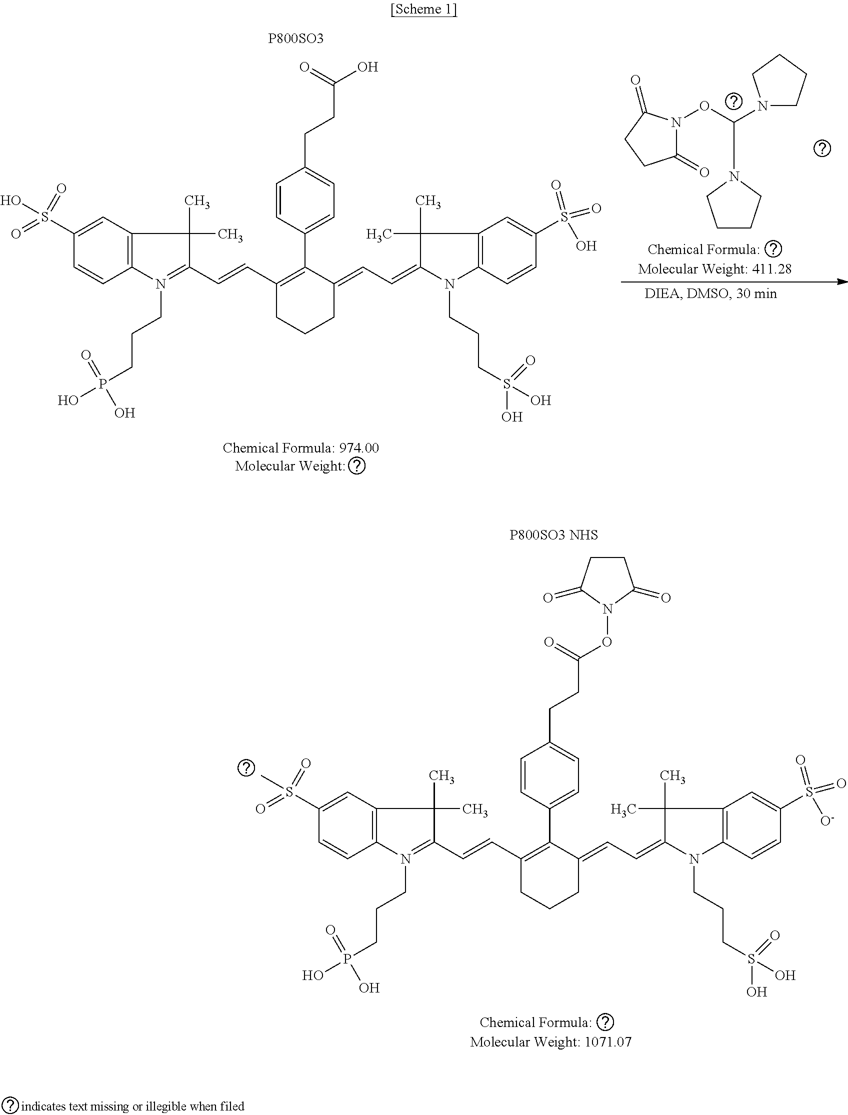

Example 1-2: Preparation of bone cell transformation recombinant protein

In order to prepare a bone cell transformation recombinant protein, Oct4 protein was used as a bone cell transformation inducing factor, and 30Kc19 protein was used as a cell permeable protein. Specifically, 30Kc19-Oct4 gene was transfected into a plasmid vector (pEt-21a), and then protein overexpression was induced through bacterial (BL21) transformation to prepare a 30Kc19-Oct4 recombinant protein. The recombinant protein produced from bacteria was purified using liquid chromatography.

Example 1-3: Preparation of complex

To prepare a complex, 1.5 mg of functional group (NHS ester)-activated P800SO3 was added to the recombinant protein dissolved in PBS to prepare a mixed solution. As the recombinant protein, 1.59 mg of 30Kc19-Oct4 dissolved in 4 ml of PBS was used. The pH was adjusted between 8 and 8.5 and gently shaken at room temperature for 2 hours. Centrifugation was performed using a 10 kDa Vivaspin column at 4000 rpm (X3) for 15 minutes. A P6 column (Bio-rad, Bio-Spin®) was used for final purification.

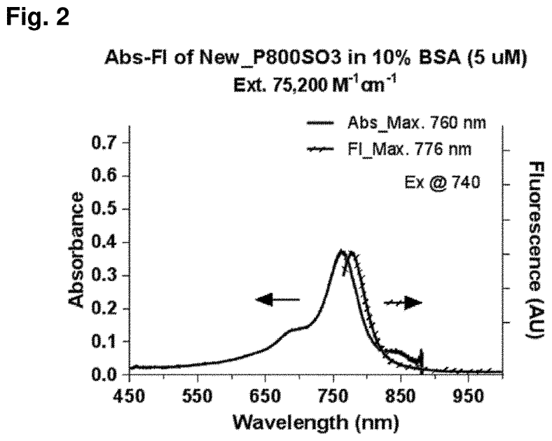

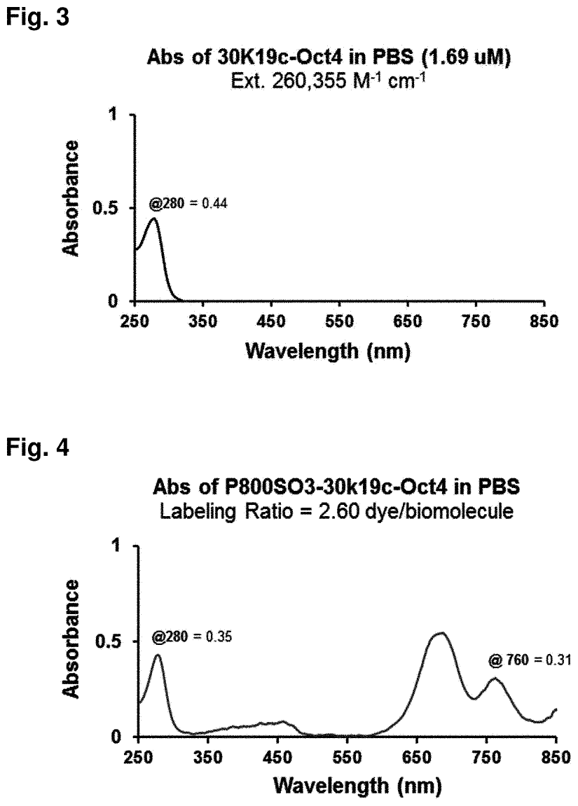

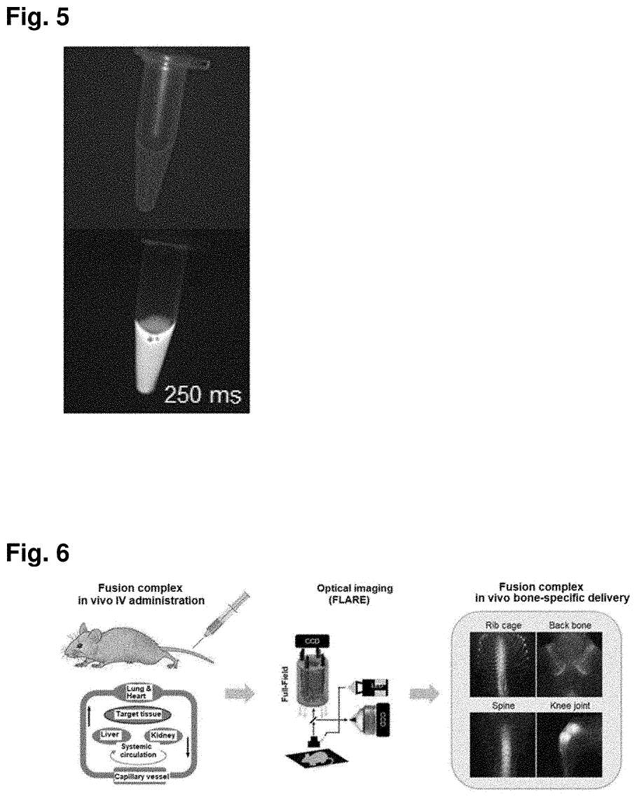

After centrifugation with the P6 column to remove water, a maximum of 100 μL of the mixed solution was added to each column. After centrifugation at 1000 (x g) for 4 minutes, a protein fusion complex was collected. Absorbance of the recombinant protein, 30K19c-Oct4 and P800SO3 was measured with a spectrophotometer. The measurement results of spectrophotometer are shown in FIGS. 2 to 4. FIG. 2 shows absorbance of P800SO3, FIG. 3 shows absorbance of 30Kc19-Oct4, and FIG. 4 shows absorbance of P800SO3-30K-Oct4. FIG. 5 is a color image and a fluorescence image obtained using an optical imaging device for P800SO3-30K-Oct4 before administration.

The synthesis conditions as described above was optimized so that an average of about 2.5 P800SO3 molecules were synthesized onto one recombinant protein molecule to prepare a P800SO3-30K-Oct4 complex. The process for preparation of the complex is shown in Scheme 2.

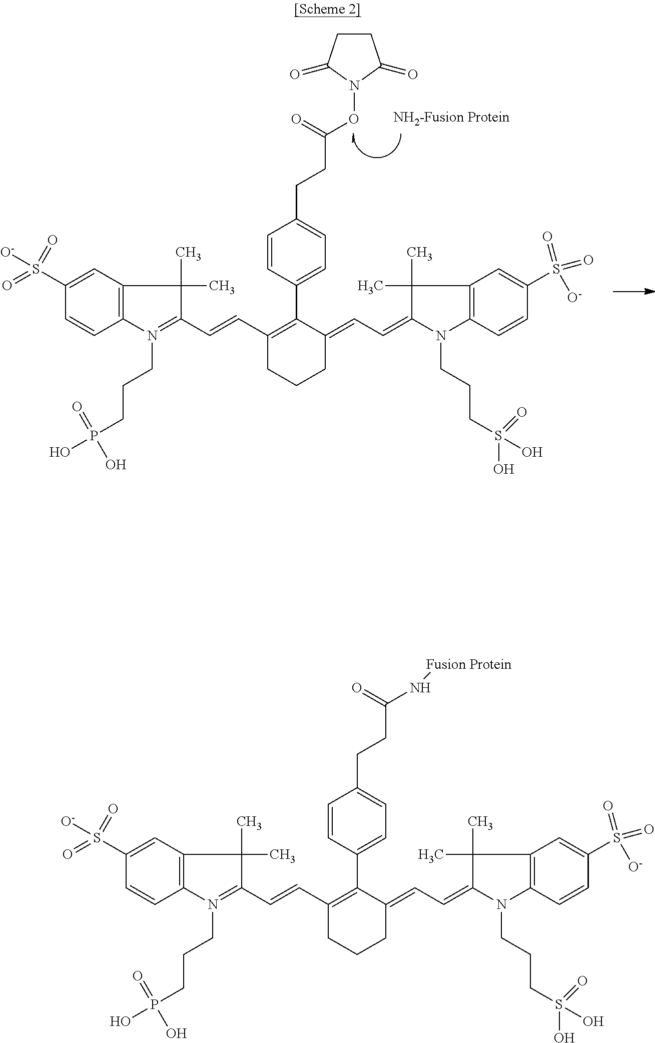

Experimental Example

100 to 150 μL of the P800SO3-30K-Oct4 complex was administered to a tail vein of a normal 6-week-old male rat model (Charles River). The experimental procedure is depicted in FIG. 6. At 4 hours, 24 hours, and 5 days after administration, rib cage, back bone, spine and knee joint were photographed with an optical imaging device to determine the biodistribution.

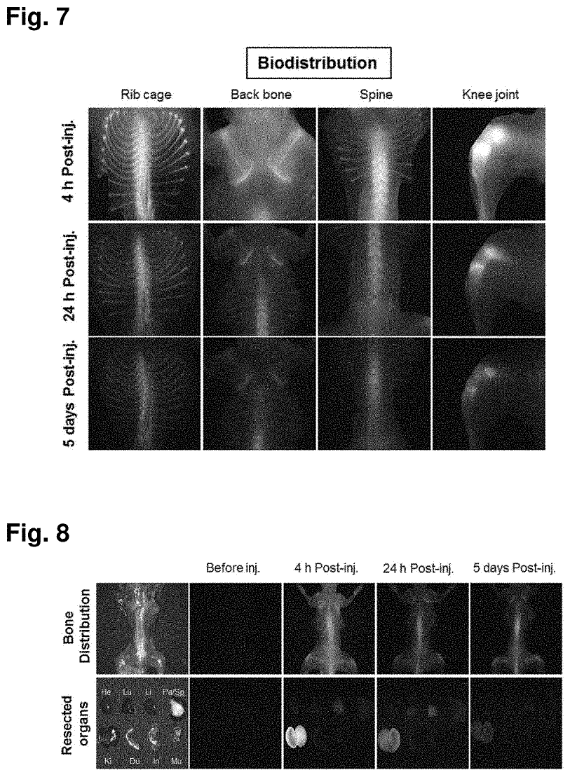

Specifically, color channel and fluorescence channel (800 nm) images were obtained by the optical imaging device, and the results are shown in FIG. 7. The exposure time in the linear signal intensity range of the fluorescent camera was 100 to 500 msec, and the intensity of the near-infrared light source on the hiosurface was maintained at >5mW/cm2. In addition, it was observed that the fluorescent materials bound to the bone tissue were strongly expressed even after 5 days.

In addition, the images by the optical imaging device, showing the biodistribution in each organ over time are shown in FIG. 8. As shown in FIGS. 7 and 8, it was found that the complex has a significantly low uptake in tissues other than bone tissue while it is specifically targeted to the bone tissue and expressed therein. Moreover, it was observed that the fluorescent materials bound to the bone tissue were strongly expressed even after 5 days.

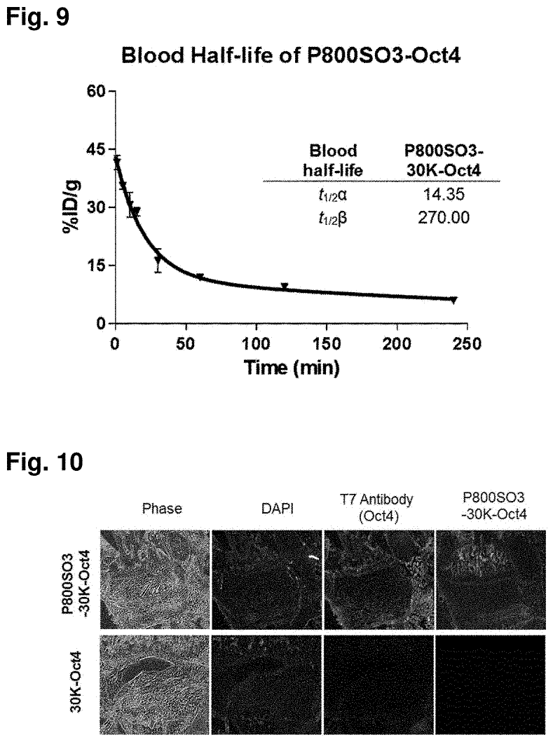

In addition, the blood half-life was determined for pharmacokinetic analysis. Specifically, the fluorescence signal of blood was measured over time, and the results are shown in FIG. 9.

In the graph of FIG. 9, t_{1/2}β denotes a decrease rate in plasma concentration of drug distribution phase, and t_{1/2}β) denotes a decrease rate of drug elimination phase due to metabolism. As shown in FIG. 9, it can be seen that the complex is discharged from the body within 24 hours.

In addition, the distribution of Oct4 and the complex in the spine was observed with DAPI (1:200, Sigma--Aldrich, USA) and T7 antibody (Abeam®) by immunohistochemistry. Specifically, a tissue specimen with a thickness of 10 μm was prepared using a microtome, a first antibody (T7) was attached to the specimen, and a secondary antibody with fluorescent (Alexa 488) was attached thereto. It was observed through a fluorescence microscope.

The results are shown in FIG. 10, and as shown in the figure, the targeted delivery to the spinal tissue of the complex was confirmed by determining the expression of Oct4. As described above, according to the present invention, it is possible to induce bone-specific differentiation through the bone targeting complex without introduction of foreign genes that requires a viral vector. The present invention can be effectively applied to bone diseases such as osteoporosis. In addition, since the delivery of the complex can be confirmed by real-time imaging, the bone cell transformation efficiency into during injection can be further improved. Furthermore, as the complex of the present invention can be. used to induce direct transdifferentiation in vivo by targeting a specific tissue, it can be applied to various disease models.

The above descriptions are merely illustrative of the technical idea of the present invention, and those of ordinary skill in the technical field to which the present invention pertains can make various modifications and variations without departing from the essential characteristics of the present invention. In addition, the embodiments disclosed in the present invention are not intended to limit the technical idea of the present invention, but to explain the technical idea, and the scope of the technical idea of the present invention is not limited by these embodiments. The scope of protection of the present invention should be interpreted by the appended claims, and all technical ideas within the scope equivalent thereto should be interpreted as being included in the scope of the present invention.

1. A bone-specific complex comprising a bone cell transformation recombinant protein and a bone-targeting molecule.

2. The bone-specific complex according to claim 1, wherein the bone cell transformation recombinant protein comprises Oct4 protein.

3. The bone-specific complex according to claim 1, wherein the bone cell transformation recombinant protein comprises a cell permeable protein.

4. The bone-specific complex according to claim 3, wherein the cell permeable protein comprises 30Kc19.

5. The bone-specific complex according to claim 1, wherein the bone-targeting molecule is a phosphonated near-infrared fluorescent substance.

6. The bone-specific complex according to claim 1, wherein the bone-targeting molecule comprises P800SO3.

7. The bone-specific complex according to claim 1, wherein the complex is P800SO3-30K-Oct4.

8. A method for producing a bone-specific complex, comprising:
binding a bone cell transformation inducing factor to a cell permeable protein to prepare a bone cell transformation recombinant protein;
succinimidyl ester (NHS ester)-activating on a bone-targeting molecule; and
binding the activated bone-targeting molecule to the recombinant protein.

9. The method for producing a bone-specific complex according to claim 8, wherein an average of 1 to 5 bone-targeting molecules are synthesized onto said recombinant protein molecule.

10. The method for producing a bone-specific complex according to claim 8, wherein:
the bone cell transformation inducing factor comprises Oct4 protein,
the cell permeable protein comprises 30Kc19, and
the bone-targeting molecule comprises P800SO3.
